(19) United States
(12) Patent Application Publication
(10) Pub. No.: US 2023/0142931 A1
(43) Pub. Date: May 11, 2023

(54) Zinc-Containing Medical Instrument

(71) Applicant: BIOTYX MEDICAL (SHENZHEN) CO., LTD., SHENZHEN, GUANGDONG (CN)

(72) Inventors: Deyuan Zhang, Shenzhen (CN); Haiping Qi, Shenzhen (CN); Wenjiao Lin, Shenzhen (CN)

(73) Assignee: BIOTYX MEDICAL (SHENZHEN) CO., LTD., SHENZHEN, GUANGDONG (CN)

(21) Appl. No.: 17/915,430

(22) PCT Filed: Apr. 7, 2021
(86) PCT No.: PCT/CN2021/000059

§ 371 Date: Sep. 28, 2022

(30) Foreign Application Priority Data
Apr. 7, 2020 (CN) CN202010266038.9

(51) Int. Cl. A61L 31/02; C08L 67/04; A61L 31/16; A61L 31/10

(52) U.S. Cl. CPC A61L 31/022; C08L 67/04; A61L 31/16; A61L 31/10

(57) ABSTRACT

The present invention relates to a zinc-containing medical device, including a zinc-containing matrix and a polylactic acid coating arranged on the zinc-containing matrix. The polylactic acid coating has a thickness of x μm; and when x and the weight-average molecular weight Mn (kDa) of polylactic acid satisfied the following formula:

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 ,$$

the corrosion rate of zinc in the matrix is relatively small, sufficient mechanical properties can be maintained within the repair period, and the biological risk is relatively low. When the polylactic acid is poly-racemic lactic acid, a=0.0336 ln(Mn)−0.1449, b=−0.472 ln(Mn)+2.1524, and c=1.1604 ln(Mn)−5.7128; and when the polylactic acid is poly-L-lactic acid, a=−0.006 ln(Mn)+0.03441, b=0.0648 ln(Mn)−0.3662, and c=−0.162 ln(Mn)+0.7847.

### TECHNICAL FIELD

The present invention relates to the field of interventional medical devices, and more particularly relates to a zinc-containing medical device.

### BACKGROUND ART

This section provides merely background information related to the present invention, which is not necessarily the existing art.

At present, the matrix materials of absorbable implantable medical devices are mainly selected from degradable polymers and corrodible metals. Among the degradable polymers, polylactic acid is the most widely used. The advantage of polylactic acid is that it can completely degrade to degradation products including carbon dioxide and water, but it has insufficient mechanical properties as its disadvantage. The advantages of corrodible metals are that they are easy to process and plastic, and have high mechanical strength. Commonly used corrodible metals in clinical settings mainly include magnesium and magnesium-based alloys, iron and iron-based alloys, and zinc and zinc-based alloys.

From the perspective of clinical application, on the one hand, the device needs to maintain structural integrity and have sufficient mechanical properties from the time of implantation until a diseased part heals and its morphology and functions return to normal. This requires that the rate of early corrosion or the rate of degradation of absorbable implantable medical devices be as slow as possible. On the other hand, the corrosion of a corrodible metal material will generate corrosion products including metal ions. Higher metal ion concentration may be toxic, resulting in biological risk. For example, it has been reported that the cytotoxic (LD50) concentrations of zinc ions to fibroblasts, smooth muscle cells and endothelial cells are 50 μmol/L, 70 μmol/L, and 265 μmol/L, respectively. Therefore, for zinc-containing medical devices, the corrosion rate of zinc must be controlled to maintain sufficient mechanical properties during the repair period and reduce the concentration of zinc ions, thereby further reducing the biological risk.

### SUMMARY OF THE INVENTION

Based on the above, it is necessary to provide a zinc-containing absorbable device that can maintain sufficient mechanical properties during the repair period with low biological risk.

A zinc-containing medical device comprises a zinc-containing matrix and a polylactic acid coating disposed on the surface of the zinc-containing matrix, and the polylactic acid coating has a thickness of x that satisfies the following formula:

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2.$$

wherein, when the polylactic acid is poly-racemic lactic acid, a=0.0336 ln(Mn)−0.1449, b=−0.472 ln(Mn)+2.1524, and c=1.1604 ln(Mn)−5.7128;
when the polylactic acid is poly-L-lactic acid, a=−0.006 ln(Mn)+0.03441, b=0.0648 ln(Mn)−0.3662, and c=−0.162 ln(Mn)+0.7847; and
the Mn is the weight-average molecular weight of the polylactic acid, which is in kDa;
and x is in μm.

In one of the embodiments, the zinc-containing matrix has an outer surface, an inner surface, and a side surface; the polylactic acid coating at least covers the outer surface or the inner surface or the side surface; the polylactic acid is poly-racemic lactic acid with a weight-average molecular weight of 100-300 kDa; and the average thickness of the part located on the outer surface of the polylactic acid coating is 5.2-11.5 μm; the average thickness of the part located on the inner surface of the polylactic acid coating is 5.2-11.5 μm; and the average thickness of the part located on the side surface of the polylactic acid coating is 5.2-11.5 μm.

In one of the embodiments, the zinc-containing matrix has an outer surface, an inner surface, and a side surface; the polylactic acid coating at least covers the outer surface or the inner surface or the side surface; the polylactic acid is poly-racemic lactic acid with an weight-average molecular weight of 10-100 kDa; and the average thickness of the part located on the outer surface of the polylactic acid coating is 2-9 Lim; the average thickness of the part located on the inner surface of the polylactic acid coating is 2-9 μm; and the average thickness of the part located on the side surface of the poly lactic acid coating is 2-9 μm.

In one of the embodiments, the zinc-containing matrix has an outer surface, an inner surface, and a side surface; the polylactic acid coating at least covers the outer surface or the inner surface or the side surface; the polylactic acid is poly-racemic lactic acid with an weight-average molecular weight of 2-10 kDa; and the average thickness of the part located on the outer surface of the polylactic acid coating is 1.5-5.5 μm; the average thickness of the part located on the inner surface of the polylactic acid coating is 1.5-5.5 μm; the average thickness of the part located on the side surface of the polylactic acid coating is 1.5-5.5 μm.

In one of the embodiments, the zinc-containing matrix has an outer surface, an inner surface, and a side surface; the polylactic acid coating at least covers the outer surface or the inner surface or the side surface; the polylactic acid is poly-L-lactic acid with an weight-average molecular weight of 200-300 kDa; and the average thickness of the part located on the outer surface of the polylactic acid coating is 9-22 μm; the average thickness of the part located on the inner surface of the polylactic acid coating is 9-22 μm; and the average thickness of the part located on the side surface of the polylactic acid coating is 9-22 μm.

In one of the embodiments, the zinc-containing matrix has an outer surface, an inner surface, and a side surface; the polylactic acid coating at least covers the outer surface or the inner surface or the side surface; the polylactic acid is poly-L-lactic acid with an weight-average molecular weight of 50-200 kDa; and the average thickness of the part located on the outer surface of the polylactic acid coating is 7-13 μm; the average thickness of the part located on the inner surface, of the polylactic acid coating is 7-13 μm; and the average thickness of the part located on the side surface of the polylactic acid coating is 7-13 μm.

In one of the embodiments, the zinc-containing matrix has an outer surface, an inner surface, and a side surface; the polylactic acid coating covers the outer surface, the inner surface, and the side surface; and the average thickness of the part located on the outer surface of the polylactic acid coating is xₒᵤₜₑᵣ; the average thickness of the part located on the inner surface of the polylactic acid coating is xᵢₙₙₑᵣ; the average thickness of the part located on the side surface of the polylactic acid coating is x_{side}; xᵢₙₙₑᵣ≤xₒᵤₜₑᵣ, xᵢₙₙₑᵣ≤x_{side}, and at least one of xₒᵤₜₑᵣ, xᵢₙₙₑᵣ, and x_{side} satisfies the following formula:

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 , where , x = {x}_{inner} , {x}_{side} or {x}_{outer} .$$

wherein, when the polylactic acid is poly-racemic lactic acid, a=0.0336 ln(Mn)−0.1449, b=−0.472 ln(Mn)+2.1524, and c=1.1604 ln(Mn)−5.7128;
when the polylactic acid is poly-L-lactic acid, a=−0.006 ln(Mn)+0.03441, b=0.0648 ln(Mn)−0.3662, and c=−0.162 ln(Mn)+0.7847; and
the Mn is the weight-average molecular weight of the polylactic acid, which is in kDa;
x is in μm.

In one of the embodiments, the material of the zinc-containing matrix is pure zinc or zinc alloy; or, the zinc-containing matrix includes a body and a zinc-containing layer attached to the body; and the material of the zinc-containing layer is pure zinc or zinc alloy.

In one of the embodiments, when the material of the zinc-containing matrix is pure zinc or zinc alloy, the mass percentage of zinc in the zinc alloy is 50%-99.99%;

when the zinc-containing matrix includes the body and the zinc-containing layer attached to the body, and the material of the zinc-containing layer is zinc alloy, the mass percentage of zinc in the zinc alloy is 50%-99.99%.

In one of the embodiments, the zinc-containing layer covers all surfaces of the body.

In one of the embodiments, the polylactic acid coating contains an active drug.

It has been proven by experiment that when the weight-average molecular weight Mn (kDa) of the polylactic acid in the polylactic acid coating and the thickness x (μm) of the polylactic acid coating satisfy the following formula

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 ,$$

and when the polylactic acid is poly-racemic lactic acid, a=0.0336 ln(Mn)−0.1449, b=−0.472 ln(Mn)+2.1524, and c=1.1604 ln(Mn)−5.7128; when the polylactic acid is poly-L-lactic acid, a=−0.006 ln(Mn)+0.03441, b=0.0648 ln(Mn)−0.3662, and c=−0.162 ln(Mn)+0.7847, the corrosion rate of zinc in the zinc-containing matrix is relatively small so as to prevent fast corrosion of the zinc-containing matrix, and can maintain sufficient mechanical properties during the repair period. Furthermore, the lower corrosion rate of the zinc in the zinc-containing matrix can avoid an extremely high cumulative concentration of zinc ions, which reduces biological risk.

### BRIEF DESCRIPTION OF THE DRAWINGS

FIG. 1 is a pathological section diagram of the tissue around the iron-based stent after the zinc-based stent of Example 5 was implanted into the rabbit iliac artery for 1 month.

### DETAILED DESCRIPTION OF THE INVENTION

In order to make the foregoing objectives, features and advantages of the present invention more obvious and understandable, the specific embodiments of the present invention will be described in detail below with reference to the accompanying drawings. In the following description, many specific details are described to provide a thorough understanding of the present invention. However, the present invention can be implemented in a variety of other ways than those described herein, and those skilled in the art can make similar improvements without departing from the connotation of the present invention. Therefore, the present invention is not limited by the specific embodiments disclosed below.

Unless otherwise defined, all technical and scientific terms used herein have the same meanings as commonly understood by those skilled in the art to which the present invention belongs. The terms used herein in the description of the present invention are merely for the purpose of describing specific embodiments and are not intended to limit the present invention.

In one embodiment, a zinc-containing medical device includes a zinc-containing matrix and a polylactic acid coating arranged on the zinc-containing matrix. Among that, the zinc-containing matrix is an absorbable matrix. The polylactic acid coating at least covers a partial surface of the zinc-containing matrix.

In one embodiment, the material of the zinc-containing matrix is pure zinc or zinc alloy, that is, the zinc-containing matrix is made of pure zinc or zinc alloy. For example, the zinc-containing matrix is a hollow lumen structure made of pure zinc or a zinc alloy. Among them, the zinc alloy is a bioabsorbable alloy.

In one embodiment, the alloying element in the zinc alloy is at least one of C, N, O, S, P, Ce, Mn, Ca, Cu, Pd, Si, W, Ti, Co, Cr, Cu, and Re. It should be noted that the alloying elements in the zinc alloy are non-toxic and harmless to living organisms. Alternatively, the content of the alloying elements is low enough not to cause toxic effects on the organisms.

In one embodiment, the mass percentage of zinc in the zinc alloy is 50%-99.99%.

In another embodiment, the zinc-containing matrix includes a body and a zinc-containing layer attached to the body, and the material of the zinc-containing layer is pure zinc or zinc alloy. The material of the body is an absorbable metal or absorbable polymer. For example, the absorbable metal is pure iron or an iron-based alloy. The zinc-containing layer at least covers a partial surface of the body. In one embodiment, the zinc-containing layer covers all surfaces of the body.

When the material of the zinc-containing layer is the zinc alloy, the alloying element in the zinc alloy is at least one of C, N, O, S, P, Ce, Mn, Ca, Cu, Pd, Si, W, Ti, Co, Cr, Cu, and Re. It should be noted that the alloying element in the zinc alloy is non-toxic and harmless to living organisms. Alternatively, the content of the alloying element is low enough not to cause toxic effects on living organisms. In one embodiment, the mass percentage of zinc in the zinc alloy is 50%-99.99%.

Whether the zinc-containing matrix is a matrix made of pure zinc or zinc alloy or the zinc-containing matrix includes a body and a zinc-containing layer attached to the body, in one embodiment, the zinc-containing matrix has an outer surface, an inner surface, and a side surface. The polylactic acid coating at least covers the outer surface of the zinc-containing matrix. Or the polylactic acid coating at least covers the inner surface of the zinc-containing matrix. Alternatively, the polylactic acid coating at least covers the side surface of the zinc-containing matrix. As defined, when the zinc-containing medical device is implanted into the body, the inner surface is the surface in direct contact with body fluids (e.g. blood), the outer surface is the surface in direct contact with the tissue walls (e.g. vascular walls), and the side surface is connected to the inner surface and outer surface.

In one embodiment, the polylactic acid coating covers the outer surface, the inner surface, and the side surface of the zinc-containing matrix.

When the zinc-containing medical device is implanted into the body, zinc corrosion can generate zinc phosphate, at the same time, the polylactic acid is degraded to generate carboxyl and hydroxyl. Zinc phosphate can react with carboxyl and hydroxyl to form complexes, thereby hindering further corrosion of the zinc. Thus, the zinc corrosion rate of the zinc-containing medical device that includes a polylactic acid coating is smaller than the zinc corrosion rate of the zinc-containing medical device that does not include a polylactic acid coating. Meanwhile, hydrogen ions generated by the degradation of the polylactic acid will accelerate the corrosion of metals. In other words, the polylactic acid coating has both inhibition effects and promotion effects on the corrosion rate of the zinc in the zinc-containing matrix. The release rate and cumulative concentration of PLA degradation products will affect the corrosion inhibition and promotion effects of the polylactic acid coating on the zinc corrosion. The properties of the polylactic acid itself in the polylactic acid coating are an important factor affecting the degradation rate of the polylactic acid. The thickness of the polylactic acid coating affects the total amount of the degradation products of the polylactic acid.

The thickness of the polylactic acid coating is x, which is in μm, and satisfies the following formula:

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2.$$

wherein, when the polylactic acid is poly-racemic lactic acid, a=0.0336 ln(Mn)−0.1449, b=−0.472 ln(Mn)+2.1524, and c=1.1604 ln(Mn)−5.7128; and
when the polylactic acid is poly-L-lactic acid, a=−0.006 ln(Mn)+0.03441, b=0.0648 ln(Mn)−0.3662, and c=−0.162 ln(Mn)+0.7847.

Among them, Mn is the weight-average molecular weight of the polylactic acid, which is in kDa. That is, when calculating a, b, and c, the numerical value of Mn is put into the formulas according to the numerical value in kDa for calculation. The calculated numerical values are directly used in the above formula or relational expression. When the thickness x of the polylactic acid coating is put into the above formula or relational expression, the corresponding numerical value in μm of the thickness of the polylactic acid coating is put into the above relational expression for comparison.

The above thickness x of the polylactic acid coating refers to the average thickness xₒᵤₜₑᵣ of the part located on the outer surface of the polylactic acid coating; the average thickness xᵢₙₙₑᵣ of the part located on the inner surface of the polylactic acid coating; or the average thickness x_{side} of the part located on the side surface of the polylactic acid coating. That is, at least one of xₒᵤₜₑᵣ, xᵢₙₙₑᵣ, and x_{side} satisfies the above formula.

In one embodiment, xᵢₙₙₑᵣ≤xₒᵤₜₑᵣ, xᵢₙₙₑᵣ≤x_{side}, and at least one of xₒᵤₜₑᵣ, xᵢₙₙₑᵣ, and x_{side} satisfies the following formula:

$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 , where , x = {x}_{inner} , {x}_{side} or {x}_{outer} .$$

In one embodiment, xₒᵤₜₑᵣ, xᵢₙₙₑᵣ and x_{side} all satisfy the above formula.

It has been proven by experiments that when the thickness of the polylactic acid coating, the type of the polylactic acid, and the molecular weight of the polylactic acid satisfy the above relationship, the corrosion inhibition effect is dominant among the effects of the polylactic acid coating on the corrosion rate of the zinc in the zinc-containing matrix, so that the corrosion rate of the zinc is relatively small.

By arranging the polylactic acid coating with the thickness of x μm on the zinc-containing matrix, and selecting the suitable polylactic acid, the corrosion rate of the zinc in the zinc-containing matrix is relatively small. When the zinc-containing matrix is a matrix made of pure zinc or zinc alloy, the corrosion rate of the zinc is relatively small, that is, the corrosion rate of the zinc-containing matrix itself is relatively small, so as to avoid fast corrosion of the zinc-containing matrix and quick loss of mechanical properties, which is therefore conducive to maintaining sufficient mechanical properties during the repair period. When the zinc-containing matrix includes a body and a zinc-containing layer attached to the body, and the material of the zinc-containing layer is pure zinc or zinc alloy, the zinc-containing layer is coated on the surface of the body to physically isolate the body and body fluids. The corrosion rate of the zinc-containing layer is relatively small, which is conducive to extending the protection period of the body, thereby maintaining sufficient mechanical properties during the repair period.

Furthermore, due to the small corrosion rate of zinc, the cumulative concentration of corrosion products of zinc in tissues is relatively low, which reduces the biological risk.

It should be noted that when the thickness x of the polylactic acid coating satisfies the above formula, the corrosion rate of the zinc in the zinc-containing matrix is relatively small, which means that the comparison is carried out under the same premise of the other conditions, such as the same zinc-containing matrix, the same polylactic acid material, and the same distribution of the polylactic acid coating. Alternatively, under the premise of the same zinc-containing matrix, the corrosion rate of zinc in a zinc-containing medical device that includes the above polylactic acid coating is smaller than that of zinc in a device which does not include the polylactic acid coating.

In one embodiment, the polylactic acid is poly-racemic lactic acid with the weight-average molecular weight of 100-300 kDa; the average thickness of the part located on the outer surface of the polylactic acid coating is 5.2-11.5 μm; and/or, the average thickness of the part located on the inner surface of the polylactic acid coating is 5.2-11.5 μm; and/or, the average thickness of the part located on the side surface of the polylactic acid coating is 5.2-11.5 μm. In one embodiment, the average thicknesses of all the surfaces of the polylactic acid coating are 5.2-11.5 μm.

In one embodiment, the polylactic acid is poly-racemic lactic acid with the weight-average molecular weight of 10-100 kDa; the average thickness of the part located on the outer surface of the polylactic acid coating is 2-9 μm; and/or, the average thickness of the part located on the inner surface of the polylactic acid coating is 2-9 μm; and/or, the average thickness of the part located on the side surface of the polylactic acid coating is 2-9 μm. In one embodiment, the average thicknesses of all the surfaces of the polylactic acid coating are 2-9 μm.

In one embodiment, the polylactic acid is poly-racemic lactic acid with the weight-average molecular weight of 2-10 kDa; the average thickness of the part located on the outer surface of the polylactic acid coating is 1.5-5.5 μm; and/or, the average thickness of the part located on the inner surface of the polylactic acid coating is 1.5-5.5 μm; and/or, the average thickness of the part located on the side surface of the polylactic acid coating is 1.5-5.5 μm. In one embodiment, the average thicknesses of all the surfaces of the polylactic acid coating are 1.5-5.5 μm.

In one embodiment, the polylactic acid is poly-racemic lactic acid with the weight-average molecular weight of 200 kDa; the average thickness of the part located on the outer surface of the polylactic acid coating is 9.1 μm; and/or, the average thickness of the part located on the inner surface of the polylactic acid coating is 9.1 μm; and/or, the average thickness of the part located on the side surface of the polylactic acid coating is 9.1 μm. In one embodiment, the average thicknesses of all the surfaces of the polylactic acid coating are 9.1 μm.

In one embodiment, the polylactic acid is poly-racemic lactic acid with the weight-average molecular weight of 50 kDa; the average thickness of the part located on the outer surface of the polylactic acid coating is 4.9 μm; and/or, the average thickness of the part located on the inner surface of the polylactic acid coating is 4.9 μm; and/or, the average thickness of the part located on the side surface of the polylactic acid coating is 4.9 μm. Alternatively, in one embodiment, the average thicknesses of all the surfaces of the polylactic acid coating are 4.9 μm.

In one embodiment, the polylactic acid is poly-racemic lactic acid with a weight-average molecular weight of 5 kDa; the average thickness of the part located on the outer surface of the polylactic acid coating is 3.6 μm; and/or, the average thickness of the part located on the inner surface of the polylactic acid coating is 3.6 μm; and/or, the average thickness of the part located on the side surface of the polylactic acid coating is 3.6 μm. In one embodiment, the average thicknesses of all the surfaces of the polylactic acid coating are 3.6 μm.

In one embodiment, the polylactic acid is poly-L-lactic acid with the weight-average molecular weight of 200-300 kDa; the average thickness of the part located on the outer surface of the polylactic acid coating is 9-22 μm; or the average thickness of the part located on the inner surface of the polylactic acid coating is 9-22 μm; or the average thickness of the part located on the side surface of the polylactic acid coating is 9-22 μm. In one embodiment, the average thicknesses of all the surfaces of the polylactic acid coating are 9-22 μm.

In one embodiment, the polylactic acid is poly-L-lactic acid with the weight-average molecular weight of 50-200 kDa; the average thickness of the part located on the outer surface of the polylactic acid coating is 7-13 μm; or the average thickness of the part located on the inner surface of the polylactic acid coating is 7-13 μm; or the average thickness of the part located on the side surface of the polylactic acid coating is 7-13 μm. In one embodiment, the average thicknesses of all the surfaces of the polylactic acid coating are 7-13 μm.

In one embodiment, the polylactic acid is poly-L-lactic acid with the weight-average molecular weight of 300 kDa; the average thickness of the part located on the outer surface of the polylactic acid coating is 19.7 μm; or the average thickness of the part located on the inner surface of the polylactic acid coating is 19.7 μm; or the average thickness of the part located on the side surface of the polylactic acid coating is 19.7 μm. In one embodiment, the average thicknesses of all the surfaces of the polylactic acid coating are 19.7 μm.

In one embodiment, the polylactic acid is poly-L-lactic acid with the weight-average molecular weight of 100 kDa; the average thickness of the part located on the outer surface of the polylactic acid coating is 9.4 μm; or the average thickness of the part located on the inner surface of the polylactic acid coating is 9.4 μm; or the average thickness of the part located on the side surface of the polylactic acid coating is 9.4 μm. In one embodiment, the average thicknesses of all the surfaces of the polylactic acid coating are 9.4 μm.

In one embodiment, the polylactic acid coating contains an active drug. In one embodiment, the active drug is selected from at least one of the drugs that inhibit vascular proliferation, anti-platelet drugs, anti-thrombotic drugs, anti-inflammatory drugs, and anti-allergic drugs. The drug that inhibits vascular proliferation is at least one of paclitaxel, rapamycin, and derivatives thereof. The anti-platelet drug may be cilostazol. The anti-thrombotic drug may be heparin. The anti-inflammatory drug may be dexamethasone. The anti-allergic drug is selected from at least one of calcium gluconate, chlorpheniramine, and cortisone. When the polylactic acid coating contains an active drug, that is, when polylactic acid is mixed with the active drug to form a mixed coating, the thickness of the polylactic acid coating can be converted from the thickness of the mixed coating according to the mass ratio of the polylactic acid to the drug: the thickness (x) of the polylactic acid coating=the thickness of the mixed coating x the mass percentage of the polylactic acid. In one embodiment, the active drug may be distributed in at least one of the outer surfaces, the inner surface, and the side surface of the zinc-containing matrix.

In one embodiment, the polylactic acid is poly-racemic lactic acid with the weight-average molecular weight of 5 kDa. The average thicknesses of the parts located on the inner surface and the side surface of the polylactic acid coating are 0, that is, the polylactic acid coating does not cover the inner surface and the side surface. The part located on the outer surface contains sirolimus; the average thickness of the polylactic acid-sirolimus coating is 5.4 μm; and the average thickness of the part located on the outer surface of the polylactic acid coating is converted to be 3.6 μm. The mass ratio of the polylactic acid to the sirolimus is 2:1.

In one embodiment, the polylactic acid is poly-racemic lactic acid with the weight-average molecular weight of 200 kDa. The average thickness of the part located on the inner surface of the polylactic acid coating is 8 microns; the parts located on the side surface and the outer surface of the polylactic acid coating both contain sirolimus, that is, the polylactic acid coating is the polylactic acid-sirolimus coating in which the mass ratio of the polylactic acid to the sirolimus is 6:1. The average thicknesses of the parts located on the side surface and the outer surface of the poly-racemic lactic acid-sirolimus coating are 10.6 μm, and the average thickness of the polylactic acid coating is converted to be 9.1 μm.

The above zinc-containing medical device may be a vascular stent, a non-vascular endoluminal stent, an occluder, an orthopedic implant, a dental implant, a respiratory implant, a gynecological implant, an andrology implant, a suture, or a bolt. The non-vascular endoluminal stent may be a tracheal stent, an esophageal stent, a urethral stent, an intestinal stent, or a biliary stent. The orthopedic implant may be a fixation screw, a fixation rivet, or a bone plate. Of course, other medical devices that need to achieve degradation and absorption can be used as the medical devices of this embodiment.

The above-mentioned zinc-containing medical devices are further described below through specific examples.

The test methods of the following examples are as follows:

1. Determination of the Weight-Average Molecular Weight of Polylactic Acid

The weight-average molecular weight is tested using a GPC-multiangle laser light scattering apparatus combined with a molecular weight test system (Wyatt, USA). The test system includes a liquid phase pump and a sample injector (Agilent, USA), an Agilent PLMIXED-C GPC column (size: 7.5×300 mm, 5 μm) (Agilent, USA), and a multi-angle laser light scattering apparatus and a differential detector (Wyatt, USA). Detection conditions are as follows:

Mobile phase: tetrahydrofuran; pump flow rate: 1 mL/min; injection volume: 100 μL; laser wavelength: 663.9 nm; and test temperature: 35° C.

2. Determination of the Thickness of the Polylactic Acid Coating

The thickness is measured using a scanning electron microscope: a sample requiring coating thickness test is fixed on a sample holder; the sample holder is then put in metal spraying equipment JFC-1600 for platinum spraying; after being sprayed once, the sample is rotated 180 degrees to be sprayed one more time to ensure all positions are sprayed; the sample sprayed with a metal on the surface is added to a Buehler room temperature resin curing agent mixed reagent prepared in a ratio of 5:1, and is made to stand for more than 8 hours before the sample can be released from a sealing shell; the sealed sample is divided equally into 3 segments; each segment is ground and polished with a semi-automatic grinding and polishing machine according to a sample polishing and grinding procedure, and a cross section of the sample to be measured shall be polished until there are no wear marks; the polished sample is fixed on an object stage of the scanning electron microscope, and the entire object stage is placed in the metal spraying equipment JFC-1600 for metal spraying for 20 seconds; and the metal-sprayed sample is placed in a scanning electron microscope JSM-6510 for thickness measurement. The thickness of the inner surface coating, the thickness of the side surface coating, and the thickness of the outer surface coating are respectively measured for each cross-section. If there are multiple rods in each cross-section, at least three rods are randomly selected to measure the thicknesses of the inner surface coatings, the thicknesses of the side surface coatings, and the thicknesses of the outer surface coatings of the rods. An average value of all the measured thicknesses of the inner surface coatings is calculated to be the average thickness of the inner surface coating of the device; the average value of all the measured thicknesses of the side surface coatings is calculated to be the average thickness of the side surface coating of the device, and the average value of all the measured thicknesses of the outer surface coatings is calculated to be the average thickness of the outer surface coating of the device.

3. Determination of the Corrosion Rate of Zinc in Zinc-Containing Medical Devices

The corrosion rate is detected by a weight loss method: a zinc-containing medical device is implanted into the body of a rabbit; and the rabbit is killed at a predetermined observation time point such as 1 month and 3 months, and the zinc-containing medical device is taken out. After the tissue on the zinc-containing medical device is carefully removed as much as possible, the zinc-containing medical device is then soaked in a saturated glycine solution and ultrasonically cleaned. After 1 min, the zinc-containing medical device is taken out of the glycine solution and immediately washed with water for 10 s. The cleaned zinc-containing medical device is soaked in 1 mol/L sodium hydroxide solution for more than 24 hours to completely dissolve zinc. The concentration of zinc in the sodium hydroxide solution is detected by atomic absorption spectrometry (AAS). The corrosion rate of zinc in the zinc-containing medical device can be obtained by calculation.

### Example 1

A zinc-based stent with a specification of 3.0 mm×8 mm included a stent matrix and a polylactic acid coating completely covering the surfaces of the stent matrix. The material of the stent matrix was pure zinc, and the stent matrix had a mass of 5 mg. Polylactic acid in the polylactic acid coating was poly-racemic lactic acid with a molecular weight of 200 kDa. The average thickness of the polylactic acid coating on an outer surface, the average thickness of the polylactic acid coating on a side surface, and the average thickness of the polylactic acid coating on an inner surface were all 9.1 μm. The polylactic acid coating was prepared by the spraying method.

### Comparative Example 1-1

A zinc-based stent with a specification of 3.0 mm×8 mm included a stent matrix and a polylactic acid coating completely covering surfaces of the stent matrix. The material of the stent matrix was pure zinc, and the stent matrix had a mass of 5 mg. Polylactic acid in the polylactic acid coating was poly-racemic lactic acid with a molecular weight of 200 kDa. The average thickness of the polylactic acid coating on an outer surface, the average thickness of the polylactic acid coating on a side surface, and the average thickness of the polylactic acid coating on an inner surface were all 6 μm. The polylactic acid coating was prepared by the spraying method.

### Comparative Example 1-2

A zinc-based stent with a specification of 3.0 mm×8 mm included a stent matrix and a polylactic acid coating completely covering surfaces of the stent matrix. The material of the stent matrix was pure zinc, and the stent matrix had a mass of 5 mg. Polylactic acid in the polylactic acid coating was poly-racemic lactic acid with the molecular weight of 200 kDa. The average thickness of the polylactic acid coating on an outer surface, the average thickness of the polylactic acid coating on a side surface, and the average thickness of the polylactic acid coating on an inner surface were all 16 The polylactic acid coating was prepared by the spraying method.

The zinc-based stents of Example 1, Comparative Example 1-1, and Comparative Example 1-2 were respectively implanted into the iliac arteries of three rabbits and were taken out after 6 months. The zinc corrosion rates were measured to be 12%, 20%, and 18%, respectively. The corrosion rate of zinc in the zinc-based stent of Example 1 is the smallest.

### Example 2

A zinc-based stent with a specification of 3.0 mm×8 mm included a stent matrix and a polylactic acid coating completely covering surfaces of the stent matrix. The material of the stent matrix was pure zinc, and the stent matrix had a mass of 5 mg. Polylactic acid in the polylactic acid coating was poly-racemic lactic acid with a molecular weight of 50 kDa. The average thickness of the polylactic acid coating on an outer surface was 6 μm; the average thickness of the polylactic acid coating on a side surface was 5.5 μm; and the average thickness of the polylactic acid coating on an inner surface was 4.9 μm. The polylactic acid coating was prepared by the spraying method.

### Comparative Example 2-1

A zinc-based stent with a specification of 3.0 mm×8 mm included a stent matrix and a polylactic acid coating completely covering surfaces of the stent matrix. the material of the stent matrix was pure zinc, and the stent matrix had a mass of 5 mg. Polylactic acid in the polylactic acid coating was poly-racemic lactic acid with a molecular weight of 50 kDa. The average thickness of the polylactic acid coating on an outer surface was 6 μm; the average thickness of the polylactic acid coating on a side surface was 5.5 μm; and the average thickness of the polylactic acid coating on an inner surface was 2.5 μm. The polylactic acid coating was prepared by the spraying method.

### Comparative Example 2-2

A zinc-based stent with a specification of 3.0 mm×8 mm included a stent matrix and a polylactic acid coating completely covering surfaces of the stent matrix. The material of the stent matrix was pure zinc, and the stent matrix had a mass of 5 mg. Polylactic acid in the polylactic acid coating was poly-racemic lactic acid with a molecular weight of 50 kDa. The average thickness of the polylactic acid coating on an outer surface was 6 μm; the average thickness of the polylactic acid coating on a side surface was 5.5 μm; and the average thickness of the polylactic acid coating on an inner surface was 8 μm. The polylactic acid coating was prepared by the spraying method.

The zinc-based stents of Example 2, Comparative Example 2-1, and Comparative Example 2-2 were respectively implanted into the iliac arteries of three rabbits and were taken out after 6 months. The zinc corrosion rates were measured to be 18%, 28%, and 24%, respectively. The corrosion rate of zinc in the zinc-based stent of Example 2 is the smallest.

### Example 3

A zinc-based stent with a specification of 3.0 mm×8 mm included a stent matrix and a polylactic acid-sirolimus coating only covering an outer surface of the stent matrix. The material of the stent matrix was pure zinc, and the stent matrix had a mass of 5 mg. Polylactic acid in the polylactic acid coating was poly-racemic lactic acid with a molecular weight of 5 kDa. The mass ratio of the poly-racemic lactic acid to the sirolimus was 2:1. The average thickness of the polylactic acid-sirolimus coating was 5.4 μm, and the average thickness of the polylactic acid coating was converted to be 3.6 μm. The polylactic acid-sirolimus coating was prepared by the 3D printing method.

### Comparative Example 3-1

A zinc-based stent with a specification of 3.0 mm×8 mm included a stent matrix. The material of the stent matrix was pure zinc, and the stent matrix had a mass of 5 mg and is basically provided with no polylactic acid coating.

### Comparative Example 3-2

A zinc-based stent with a specification of 3.0 mm×8 mm included a stent matrix and a polylactic acid-sirolimus coating only covering an outer surface of the stent matrix. The material of the stent matrix was pure zinc, and the stent matrix had a mass of 5 mg. Polylactic acid in the polylactic acid coating was poly-racemic lactic acid with a molecular weight of 5 kDa. The mass ratio of the poly-racemic lactic acid to the sirolimus was 2:1. The average thickness of the polylactic acid-sirolimus coating was 12 μm, and the average thickness of the polylactic acid coating was converted to be 8 μm. The polylactic acid-sirolimus coating was prepared by the spraying method.

The zinc-based stents of Example 3, Comparative Example 3-1, and Comparative Example 3-2 were respectively implanted into the iliac arteries of three rabbits and were taken out after 3 months. The zinc corrosion rates were measured to be 11%, 17%, and 13%, respectively. The corrosion rate of zinc in the zinc-based stent of Example 3 is the smallest.

### Example 4

A zinc-based stent with a specification of 3.0 mm×8 mm included a stent matrix and a polylactic acid coating completely covering the surfaces of the stent matrix. The material of the stent matrix was a zinc alloy, and the stent matrix had a mass of 5 mg. Polylactic acid in the polylactic acid coating was poly-l-lactic acid with a molecular weight of 300 kDa. The average thickness of the polylactic acid coating on an outer surface, the average thickness of the polylactic acid coating on a side surface, and the average thickness of the polylactic acid coating on an inner surface were all 19.6 μm. The polylactic acid coating was prepared by the spraying method.

### Comparative Example 4-1

A zinc-based stent with a specification of 3.0 mm×8 mm included a stent matrix and a polylactic acid coating completely covering the surfaces of the stent matrix. The material of the stent matrix was pure zinc, and the stent matrix had a mass of 5 mg. Polylactic acid in the polylactic acid coating was poly-l-lactic acid with a molecular weight of 300 kDa. The average thickness of the polylactic acid coating on an outer surface, the average thickness of the polylactic acid coating on a side surface, and the average thickness of the polylactic acid coating on an inner surface were all 10 pin. The polylactic acid coating was prepared by the spraying method.

### Comparative Example 4-2

A zinc-based stent with a specification of 3.0 mm×8 mm included a stent matrix and a polylactic acid coating completely covering the surfaces of the stent matrix. The material of the stent matrix was pure zinc, and the stent matrix had a mass of 5 mg. Polylactic acid in the polylactic acid coating was poly-l-lactic acid with a molecular weight of 300 kDa. The average thickness of the polylactic acid coating on an outer surface, the average thickness of the polylactic acid coating on a side surface, and the average thickness of the polylactic acid coating on an inner surface were all 30 The polylactic acid coating was prepared by the spraying method.

The zinc-based stents of Example 4, Comparative Example 4-1, and Comparative Example 4-2 were respectively implanted into the iliac arteries of three rabbits and were taken out after 6 months. The zinc corrosion rates were measured to be 21%, 29%, and 27%, respectively. The corrosion rate of zinc in the zinc-based stent of Example 4 is the smallest.

### Example 5

An iron-based stent with a specification of 3.0 mm×8 mm included a stent body made of an iron-based alloy, a pure zinc layer completely covering the surface of the stent body, and a polylactic acid-sirolimus coating covering the surface of the pure zinc layer. The mass of iron in the stent body was 4 mg; the zinc layer had a mass of 250 μg; polylactic acid in the coating was poly-racemic lactic acid with a molecular weight of 200 kDa. The mass ratio of the poly-racemic lactic acid-sirolimus coating on an inner surface was 10:1. The thickness of the poly-racemic lactic acid-sirolimus coating on the inner surface was 5.5 μm, and the thickness of the polylactic acid coating was converted to be 5 μm. Mass ratios of the poly-racemic lactic acid-sirolimus coatings on a side surface and an outer surface were 6:1. Thicknesses of the poly-racemic lactic acid-sirolimus coatings on the side surface and the outer surface were 10.6 μm, and the thickness of the polylactic acid coating was converted to be 9.1 μm. The polylactic acid coating and the polylactic acid-sirolimus coating were prepared by the spraying method.

### Comparative Example 5-1

An iron-based stent with a specification of 3.0 mm×8 mm included a stent body made of an iron-based alloy, a pure zinc layer completely covering the surface of the stent body, and a polylactic acid-sirolimus coating covering the surface of the pure zinc layer. The mass of iron in the stent body was 4 mg; the zinc layer had a mass of 250 μg; polylactic acid in the coating was poly-racemic lactic acid with the molecular weight of 200 kDa. The mass ratio of the poly-racemic lactic acid-sirolimus coating on an inner surface was 10:1. The thickness of the poly-racemic lactic acid-sirolimus coating on the inner surface was 5.5 μm, and the thickness of the polylactic acid coating was converted to be 5 μm. Mass ratios of the poly-racemic lactic acid-sirolimus coatings on a side surface and an outer surface were 6:1. Thicknesses of the poly-racemic lactic acid-sirolimus coatings on the side surface and the outer surface were 7 μm, and the thickness of the polylactic acid coating was converted to be 6 μm. The polylactic acid coating and the polylactic acid-sirolimus coating were prepared by the spraying method.

### Comparative Example 5-2

An iron-based stent with a specification of 3.0 mm×8 mm included a stent body made of an iron-based alloy, a pure zinc layer completely covering the surface of the stent body, and a polylactic acid-sirolimus coating covering the surface of the pure zinc layer. The mass of iron in the stent body was 4 mg; the zinc layer had a mass of 250 μg; polylactic acid in the coating was poly-racemic lactic acid with a molecular weight of 200 kDa. The mass ratio of the poly-racemic lactic acid-sirolimus coating on an inner surface was 10:1. The thickness of the poly-racemic lactic acid-sirolimus coating on the inner surface was 5.5 μm, and the thickness of the polylactic acid coating was converted to be 5 μm. Mass ratios of the poly-racemic lactic acid-sirolimus coatings on a side surface and an outer surface were 6:1. Thicknesses of the poly-racemic lactic acid-sirolimus coatings on the side surface and the outer surface were 17.5 μm, and the thickness of the polylactic acid coating was converted to be 15 μm. The polylactic acid coating and the polylactic acid-sirolimus coating were prepared by the spraying method.

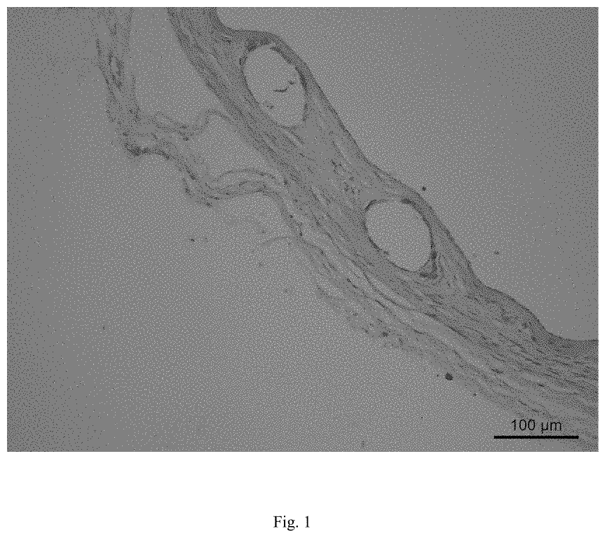

The iron-based stents with Example 5, Comparative Example 5-1, and Comparative Example 5-2 were respectively implanted into the iliac arteries of three rabbits and were taken out after 1 month. The zinc corrosion rates were measured to be 30%, 56%, and 44%, respectively. The corrosion rate of zinc in the iron-based stent of Example 5 was the smallest. It can be seen from the pathological section shown in FIG. 1 that the tissue around the zinc-based stent of Example 5 is infiltrated by a few of inflammatory cells, and there is no obvious abnormal change such as tissue necrosis.

### Example 6

An iron-based stent with a specification of 3.0 mm×8 mm included a stent body made of an iron-based alloy, a pure zinc layer completely covering the surface of the stent body, a polylactic acid coating covering an inner surface of the pure zinc layer, and a polylactic acid-sirolimus coating covering a side surface and an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on the inner surface was 6 μm. Mass ratios of the poly-racemic lactic acid to the sirolimus on the side surface and the outer surface were 3:1. Thicknesses of the poly-racemic lactic acid-sirolimus coatings on the side surface and the outer surface were 14.7 μm, and the thickness of the polylactic acid coating was converted to be 11 μm.

### Comparative Example 6-1

An iron-based stent with a specification of 3.0 mm×8 mm included a stent body made of an iron-based alloy, a pure zinc layer completely covering the surface of the stent body, a polylactic acid coating covering an inner surface of the pure zinc layer, and a polylactic acid-sirolimus coating covering a side surface and an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on the inner surface was 6 μm. Mass ratios of the poly-racemic lactic acid to the sirolimus on the side surface and the outer surface were 3:1. Thicknesses of the poly-racemic lactic acid-sirolimus coatings on the side surface and the outer surface were 8 μm, and the thickness of the polylactic acid coating was converted to be 6 μm. The polylactic acid-sirolimus coating was prepared by the spraying method.

### Comparative Example 6-2

An iron-based stent with a specification of 3.0 mm×8 mm included a stent body made of an iron-based alloy, a pure zinc layer completely covering the surface of the stent body, a polylactic acid coating covering an inner surface of the pure zinc layer, and a polylactic acid-sirolimus coating covering a side surface and an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on the inner surface was 6 μm. Mass ratios of the poly-racemic lactic acid to the sirolimus on the side surface and the outer surface were 3:1. Thicknesses of the poly-racemic lactic acid-sirolimus coatings on the side surface and the outer surface were 21 μm, and the thickness of the polylactic acid coating was converted to be 15.8 μm. The polylactic acid coating and the polylactic acid-sirolimus coating were prepared by the spraying method.

The iron-based stents of Example 6, Comparative Example 6-1, and Comparative Example 6-2 were respectively implanted into the iliac arteries of three rabbits and were taken out after 1 month. The zinc corrosion rates were measured to be 33%, 54%, and 49%, respectively. The corrosion rate of zinc in the iron-based stent of Example 6 is the smallest.

### Example 7

An iron-based stent with a specification of 3.0 mm×8 mm included a stent body made of an iron-based alloy, a pure zinc layer completely covering the surface of the stent body, a polylactic acid coating covering an inner surface and a side surface of the pure zinc layer, and a polylactic acid-sirolimus coating covering an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thicknesses of the poly-racemic lactic acid coatings on the inner surface and the side surface were 4.5 μm. The mass ratio of the poly-racemic lactic acid to the sirolimus on the outer surface was 4:1, the average thickness was 8.8 μm, and the thickness of the polylactic acid coating was converted to be 7.1 μm. The polylactic acid coating and the polylactic acid-sirolimus coating were prepared by the spraying method.

### Comparative Example 7-1

An iron-based stent with a specification of 3.0 mm×8 mm included a stent body made of an iron-based alloy, a pure zinc layer completely covering the surface of the stent body, a polylactic acid coating covering an inner surface and a side surface of the pure zinc layer, and a polylactic acid-sirolimus coating covering an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thicknesses of the poly-racemic lactic acid coatings on the inner surface and the side surface were 4.5 μm. The mass ratio of the poly-racemic lactic acid to the sirolimus on the outer surface was 4:1, the average thickness was 6 μm, and the thickness of the polylactic acid coating was converted to be 4.8 μm. The polylactic acid coating and the polylactic acid-sirolimus coating were prepared by the spraying method.

### Comparative Example 7-2

An iron-based stent with a specification of 3.0 mm×8 mm included a stent body made of an iron-based alloy, a pure zinc layer completely covering the surface of the stent body, a polylactic acid coating covering an inner surface and a side surface of the pure zinc layer, and a polylactic acid-sirolimus coating covering an outer surface of the pure zinc layer. The mass of iron in the stent body was 4 mg; the zinc layer had a mass of 250 μg; the polylactic acid in the coating was poly-racemic lactic acid with a molecular weight of 200 kDa. The average thicknesses of the poly-racemic lactic acid coatings on the inner surface and the side surface were 4.5 μm. The mass ratio of the poly-racemic lactic acid to the sirolimus on the outer surface was 4:1, the average thickness was 14.5 μm, and the thickness of the polylactic acid coating was converted to be 11.6 μm. The polylactic acid coating and the polylactic acid-sirolimus coating were prepared by the spraying method.

The iron-based stents of Example 7, Comparative Example 7-1, and Comparative Example 7-2 were respectively implanted into the iliac arteries of three rabbits and were taken out after 1 month. The zinc corrosion rates were measured to be 51%, 61%, and 63%, respectively. The corrosion rate of zinc in the iron-based stent of Example 7 is the smallest.

### Example 8

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on an inner surface was 5.1 μm, and average thicknesses of the polylactic acid coatings on an outer surface and a side surface were 7.1 μm. The polylactic acid coating was prepared by the spraying method.

### Comparative Example 8-1

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on an inner surface was 5.1 μm, and the average thicknesses of the polylactic acid coatings on an outer surface and a side surface were 6.3 μm. The polylactic acid coating was prepared by the spraying method.

### Comparative Example 8-2

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on an inner surface was 5.1 μm, and the average thicknesses of the polylactic acid coatings on an outer surface and a side surface were 14.8 μm. The polylactic acid coating was prepared by the spraying method.

The iron-based stents of Example 8, Comparative Example 8-1, and Comparative Example 8-2 were respectively implanted into the iliac arteries of three rabbits and were taken out after 1 month. The zinc corrosion rates were measured to be 48%, 54%, and 66%, respectively. The corrosion rate of zinc in the iron-based stent of Example 8 is the smallest.

### Example 9

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on an inner surface was 7.1 μm, and the average thicknesses of the polylactic acid coatings on an outer surface and a side surface were 11.1 μm. The polylactic acid coating was prepared by the spraying method.

### Comparative Example 9-1

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 200 kDa. The average thickness of the poly-racemic lactic acid coating on an inner surface was 7.1 μm, and the average thicknesses of the polylactic acid coatings on an outer surface and a side surface were 6.7 μm. The polylactic acid coating was prepared by the spraying method.

### Comparative Example 9-2

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; polylactic acid in the coating was poly-racemic lactic acid with a molecular weight of 200 kDa. The average thickness of the poly-racemic lactic acid coating on an inner surface was 7.1 μm, and the average thicknesses of the polylactic acid coatings on an outer surface and a side surface were 16.6 μm. The polylactic acid coating was prepared by the spraying method.

The iron-based stents of Example 9, Comparative Example 9-1, and Comparative Example 9-2 were respectively implanted into the iliac arteries of three rabbits and were taken out after 1 month. The zinc corrosion rates were measured to be 44%. 64%, and 62%, respectively. The corrosion rate of zinc in the iron-based stent of Example 9 is the smallest.

### Example 10

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; polylactic acid in the coating was poly-racemic lactic acid with a molecular weight of 100 kDa. The average thicknesses of the polylactic acid coatings on an inner surface, an outer surface, and a side surface were all 7.3 μm. The polylactic acid coating was prepared by the spraying method.

### Comparative Example 10-1

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 100 kDa. The average thicknesses of the polylactic acid coatings on an inner surface, an outer surface, and a side surface were all 4 μm. The polylactic acid coating was prepared by the spraying method.

### Comparative Example 10-2

An iron-based stent with a specification of 3.0 mm×8 mm included a stent body made of an iron-based alloy and a polylactic acid coating completely covering the surface of the stent matrix. The mass of iron in the stent body was 4 mg; the zinc layer had a mass of 250 μg; the polylactic acid in the coating was poly-racemic lactic acid with a molecular weight of 100 kDa. The average thicknesses of the polylactic acid coatings on an inner surface, an outer surface, and a side surface were all 11 μm. The polylactic acid coating was prepared by the spraying method.

The iron-based stents of Example 10, Comparative Example 10-1, and Comparative Example 10-2 were respectively implanted into the iliac arteries of three rabbits and were taken out after 1 month. The zinc corrosion rates were measured to be 48%, 69%, and 55%, respectively. The corrosion rate of zinc in the iron-based stent of Example 10 is the smallest.

The technical features of the examples described above can be arbitrarily combined. For the sake of brevity, not all possible combinations of the technical features of the above-described embodiments are described. However, the combinations of these technical features should be considered to be within the scope described in this specification as long as there is no contradiction in them.

The above examples only express several embodiments of the present invention, and their descriptions are more specifically and detailed, but they cannot be understood as limiting the patent scope of the present invention. It should be noted that those of ordinary skill in the art can further make various transformations and improvements without departing from the concept of the present invention, and these transformations and improvements all fall within the scope protection of the present invention. Therefore, the protection scope of the patent of the present invention shall be subject to the appended claims.

1. A zinc-containing medical device, characterized by comprising a zinc-containing matrix and a polylactic acid coating arranged on the surface of the zinc-containing matrix, wherein the polylactic acid coating has a thickness of x that satisfies the following formula:
$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 ,$$
wherein, when the polylactic acid is poly-racemic lactic acid, a=0.0336 ln(Mn)−0.1449, b=−0.472 ln(Mn)+2.1524, and c=1.1604 ln(Mn)−5.7128;
when the polylactic acid is poly-L-lactic acid, a=−0.006 ln(Mn)+0.03441, b=0.0648 ln(Mn)−0.3662, and c=−0.162 ln(Mn)+0.7847; and
Mn is the weight-average molecular weight of the polylactic acid and is in kDa; and x is in μm.

2. The zinc-containing medical device according to claim 1, characterized in that the zinc-containing matrix has an outer surface, an inner surface, and a side surface; the polylactic acid coating at least covers the outer surface or the inner surface or the side surface; the polylactic acid is poly-racemic lactic acid with a weight-average molecular weight of 100-300 kDa; the average thickness of the part located on the outer surface of the polylactic acid coating is 5.2-11.5 μm; the average thickness of the part located on the inner surface of the polylactic acid coating is 5.2-11.5 μm; and the average thickness of the part located on the side surface of the polylactic acid coating is 5.2-11.5 μm.

3. The zinc-containing medical device according to claim 1, characterized in that the zinc-containing matrix has an outer surface, an inner surface, and a side surface; the polylactic acid coating at least covers the outer surface or the inner surface or the side surface; the polylactic acid is poly-racemic lactic acid with a weight-average molecular weight of 10-100 kDa; the average thickness of the part located on the outer surface of the polylactic acid coating is 2-9 μm; the average thickness of the part located on the inner surface of the polylactic acid coating is 2-9 μm; and the average thickness of the part located on the side surface of the polylactic acid coating is 2-9 μm.

4. The zinc-containing medical device according to claim 1, characterized in that the zinc-containing matrix has an outer surface, an inner surface, and a side surface; the polylactic acid coating at least covers the outer surface or the inner surface or the side surface; the polylactic acid is poly-racemic lactic acid with a weight-average molecular weight of 2-10 kDa; the average thickness of the part located on the outer surface of the polylactic acid coating is 1.5-5.5 μm; the average thickness of the part located on the inner surface of the polylactic acid coating is 1.5-5.5 μm; and the average thickness of the part located on the side surface of the polylactic acid coating is 1.5-5.5 μm.

5. The zinc-containing medical device according to claim 1, characterized in that the zinc-containing matrix has an outer surface, an inner surface, and a side surface; the polylactic acid coating at least covers the outer surface or the inner surface or the side surface; the polylactic acid is poly-L-lactic acid with a weight-average molecular weight of 200-300 kDa; the average thickness of the part located on the outer surface of the polylactic acid coating is 9-22 μm; the average thickness of the part located on the inner surface of the polylactic acid coating is 9-22 μm; and the average thickness of the part located on the side surface of the polylactic acid coating is 9-22 μm.

6. The zinc-containing medical device according to claim 1, characterized in that the zinc-containing matrix has an outer surface, an inner surface, and a side surface; the polylactic acid coating at least covers the outer surface or the inner surface or the side surface; the polylactic acid is poly-L-lactic acid with a weight-average molecular weight of 50-200 kDa; the average thickness of the part located on the outer surface of the polylactic acid coating is 7-13 μm; the average thickness of the part located on the inner surface of the polylactic acid coating is 7-13 μm; and the average thickness of the part located on the side surface of the polylactic acid coating is 7-13 μm.

7. The zinc-containing medical device according to claim 1, characterized in that the zinc-containing matrix has an outer surface, an inner surface, and a side surface; the polylactic acid coating covers the outer surface, the inner surface, and the side surface; the average thickness of the part located on the outer surface of the polylactic acid coating is xₒᵤₜₑᵣ; the average thickness of the part located on the inner surface of the polylactic acid coating is xᵢₙₙₑᵣ; the average thickness of the part located on the side surface of the polylactic acid coating is x_{side}, xᵢₙₙₑᵣ≤xₒᵤₜₑᵣ, xᵢₙₙₑᵣ≤x_{side}, and at least one of xₒᵤₜₑᵣ, xᵢₙₙₑᵣ, and x_{side} satisfies the following formula:
$$\begin{matrix}( - b + \sqrt{{b}^{2} - 4 a c} ) \\ \end{matrix} / \begin{matrix} \\ 2 a\end{matrix} - 2 \leq x \leq \begin{matrix}( - b + \sqrt{{b}^{2} - 4 a c} ) \\ \end{matrix} / \begin{matrix} \\ 2 a\end{matrix} + 2 , where , x = {x}_{inner} , {x}_{side} or {x}_{outer} ;$$
wherein, when the polylactic acid is poly-racemic lactic acid, a=0.0336 ln(Mn)−0.1449, b=−0.472 ln(Mn)+2.1524, and c=1.1604 ln(Mn)−5.7128;
when the polylactic acid is poly-L-lactic acid, a=−0.006 ln(Mn)±0.03441, b=0.0648 ln(Mn)−0.3662, and c=−0.162 ln(Mn)+0.7847; and
Mn is the weight-average molecular weight of the polylactic acid and is in kDa; and x is in μm.

8. The zinc-containing medical device according to claim 1, characterized in that a material of the zinc-containing matrix is pure zinc or zinc alloy; or, the zinc-containing matrix comprises a body and a zinc-containing layer attached to the body; and the material of the zinc-containing layer is pure zinc or zinc alloy.

9. The zinc-containing medical device according to claim 8, characterized in that:
when the material of the zinc-containing matrix is pure zinc or zinc alloy, the mass percentage of zinc in the zinc alloy is 50%-99.99%; and
when the zinc-containing matrix comprises the body and the zinc-containing layer attached to the body, and the material of the zinc-containing layer is the zinc alloy, the mass percentage of zinc in the zinc alloy is 50%-99.99%.

10. The zinc-containing medical device according to claim 8, characterized in that the zinc-containing layer covers all surfaces of the body.

11. The zinc-containing medical device according to claim 1, characterized in that the polylactic acid coating contains an active drug.
